(19) United States
(12) Patent Application Publication
(10) Pub. No.: US 2026/0125630 A1
(43) Pub. Date: May 7, 2026

(54) BIOREACTOR

(71) Applicant: AMOGREENTECH CO., LTD, Gimpo-si (KR)

(72) Inventors: Song-Hee KOO, Gimpo-si (KR); Su-Yeon LEE, Gimpo-si (KR); Hee Sung PARK, Gimpo-si (KR); Hyeong Tak NOH, Gimpo-si (KR); Ji Young KIM, Gimpo-si (KR)

(73) Assignee: AMOGREENTECH CO., LTD, Gimpo-si (KR)

(21) Appl. No.: 19/122,783

(22) PCT Filed: Oct. 19, 2023
(86) PCT No.: PCT/KR2023/016260

§ 371 Date: Apr. 21, 2025

(30) Foreign Application Priority Data
Oct. 21, 2022 (KR) 10-2022-0136293

(51) Int. Cl. C12M 1/107; C12M 1/00; C12M 1/12; C12M 3/00

(52) U.S. Cl. CPC C12M 23/36; C12M 23/20; C12M 23/34; C12M 23/46; C12M 23/48; C12M 29/20; C12M 37/04

(57) ABSTRACT

A bioreactor for culturing/proliferating cells or acquiring exosomes secreted from the cells includes: a housing in the shape of a container having an inner space which is filled with a predetermined amount of a solution; a support assembly which includes a plurality of supports formed of a plate-shaped member having a predetermined area and spaced apart from each other in one direction, and which is disposed in the inner space to be immersed in the solution; and a gas supply part which includes a main body having a gas storage space filled with gas supplied from the outside, and is disposed in the inner space to supply gas from the gas storage space to the solution, wherein the main body is immersed in the solution filled in the inner space so that the gas stored in the gas storage space can move to the solution and be dissolved.

### CROSS REFERENCE TO THE RELATED APPLICATIONS

This application is the national phase entry of International Application No. PCT/KR2023/016260, filed on Oct. 19, 2023, which is based upon and claims priority to Korean Patent Application No. 10-2022-0136293, filed on Oct. 21, 2022, the entire contents of which are incorporated herein by reference.

### TECHNICAL FIELD

The present invention relates to a bioreactor.

### BACKGROUND

Bioprocess refers to a process of producing a desired therapeutic agent by using living cells in the biotechnology field. Antibodies, stem cells and immune cells are produced through cell culture, and these are used to produce biopharmaceuticals, vaccines and cell therapeutic agents.

In addition, exosomes are substances that exchange signals between cells and are naturally secreted from cells during the process of cell proliferation/culture. Since these exosomes contain various information such as proteins and DNA of parent cells, they are being developed as biomarkers or therapeutic agents by utilizing the same.

Meanwhile, cells are classified according to their adhesion ability into adherent cells, which must adhere to a surface substrate, and floating cells, which proliferate without attachment to a surface of a substrate.

In other words, adherent cells are cultured while being attached to a support that serves as a surface substrate, but floating cells are not cultured while being attached to a support, but rather grow while receiving signals by repeating a process of contact-floating-contact-floating, in which they attach to a surface of the support, detach, attach to the surface of the support again, and detach therefrom.

A bioreactor is used to culture these cells or obtain exosomes that are naturally secreted from the cells, and the internal environment in which cells are placed in a bioreactor must be maintained as an appropriate environment such that cells can be cultured/proliferated smoothly and cells can secrete exosomes smoothly.

In other words, the medium that supplies nutrients when culturing cells in a bioreactor must maintain a certain PH state that is suitable for cell growth, and exosomes can also be secreted from cells in larger quantities when the conditions are similar to those under which cells are cultured/proliferated.

For this purpose, a method is being used to promote smooth cell growth and exosome secretion by continuously supplying an appropriate gas to the medium or buffer solution during cell culture/proliferation.

However, if air bubbles are included in the process of supplying gas to a medium or buffer solution, cells may not be able to smoothly receive nutrients included in the medium due to the air bubbles, growth may be impaired due to stress caused by the air bubbles, or exosome secretion may not be smooth.

### SUMMARY

### Technical Problem

The present invention has been devised in consideration of the above points, and an object of the present invention is to provide a bioreactor that can smoothly supply gas to the solution while suppressing the generation of air bubbles during gas supply.

In addition, another object of the present invention is to provide a bioreactor that can easily change the oxygen concentration of a supply gas.

### Technical Solution

In order to solve the above-described problems, the present invention provides a bioreactor for culturing/proliferating cells or acquiring exosomes secreted from the cells, the bioreactor including a housing in the shape of a container having an inner space that is filled with a predetermined amount of a solution; a support assembly that includes a plurality of supports formed of a plate-shaped member having a predetermined area and spaced apart from each other in one direction, and is disposed in the inner space to be immersed in the solution; and a gas supply part that includes a main body having a gas storage space filled with gas supplied from the outside, and is disposed in the inner space to supply gas from the gas storage space to the solution, wherein the main body is disposed to be immersed in the solution filled in the inner space such that the gas stored in the gas storage space moves to the solution and is dissolved.

In addition, the gas supply part may include a main body having a gas storage space filled with gas supplied from the outside, an opening formed in the main body so as to communicate with the gas storage space, a plate-shaped porous member surrounding the main body to cover the opening, a gas inlet provided in the main body so as to allow gas to flow into the gas storage space, and a gas outlet provided in the main body so as to allow gas in the gas storage space to be discharged to the outside.

In addition, the plate-shaped porous member may be provided with a hydrophobic membrane so as to allow the gas to move from the gas storage space to the solution through the opening while blocking a solution in the inner space from moving to the gas storage space.

In addition, the bioreactor may further include a sterilization filter that is connected to the gas inlet.

In addition, the gas supply part may include a plurality of openings that are spaced apart along a peripheral direction of the main body, and the openings may be formed in the main body to have a predetermined area.

In addition, the gas supply part may be disposed in the inner space such that the main body is positioned at an upper part of the support assembly.

In addition, the gas supply part may be coupled to the housing such that a height of the main body is adjusted in the inner space.

In addition, the support may include a plate-shaped support member having a predetermined area, and a pair of nanofiber membranes that are respectively attached to both surfaces of the support member via an adhesive layer, and the nanofiber membrane may be a motif-coated plate-shaped nanofiber membrane.

In addition, the support may include a plurality of penetration holes formed through the support member such that gas introduced into the inner space from the gas supply part passes smoothly.

In addition, the support assembly may include an upper plate and a lower plate having a predetermined area; a plurality of supports disposed such that one surface faces each other between the upper plate and the lower plate; a spacer disposed between two supports facing each other so as to space the two supports apart; and a plurality of fastening bars that fasten and integrate the upper plate, the lower plate, the plurality of supports and the spacer.

In addition, the housing may include a housing body in the shape of a container having an inner space with an open upper part, and a cover configured to cover the open upper part of the housing body, and at least any one of the plurality of fastening bars may be positioned in the inner space such that a lower end contacts a bottom surface of the inner space and an upper end contacts an inner surface of the cover, thereby preventing the support assembly from floating in the inner space.

In addition, the bioreactor may further include a port formed in the housing so as to supply a solution to the inner space or discharge a solution in the inner space to the outside, and a vent formed in the housing so as to communicate with the inner space.

In addition, the gas may be a mixed gas with an oxygen concentration of 2 to 14%.

In addition, the solution may be a medium or buffer solution.

### Advantageous Effects

According to the present invention, gas with air bubbles removed can be supplied to a medium or buffer solution through a gas supply part. Through this, by using the bioreactor of the present invention, not only stable cell culture is possible, but also the amount of exosome secretion can be increased.

In addition, according to the present invention, the oxygen concentration of gas supplied through the gas supply part can be easily adjusted, thereby making it possible to culture cells or produce exosomes in an environment similar to the human body.

### BRIEF DESCRIPTION OF THE DRAWINGS

FIG. 1 is a diagram showing a bioreactor according to an embodiment of the present invention.

FIG. 2 is a view separating the main components in FIG. 1.

FIG. 3 is a cross-sectional view taken along line A-A of FIG. 1, showing a state in which the inner space is filled with a medium.

FIG. 4 is a cross-sectional view taken in the B-B direction of FIG. 1, showing a state in which the inner space is filled with a medium.

FIG. 5 is an exploded view of a support assembly that can be applied to a bioreactor according to an embodiment of the present invention.

FIG. 6 is a partial cutaway view showing the detailed lamination constitution of a support that can be used in a bioreactor according to an embodiment of the present invention.

FIG. 7 is a view showing another form of a support assembly that can be applied to a bioreactor according to an embodiment of the present invention.

FIG. 8 is a view showing a state in which the blocking member in FIG. 7 is separated.

FIG. 9 is a view showing the support assembly of FIG. 7 inserted into the housing body.

FIG. 10 is a diagram showing a gas supply part that can be applied to a bioreactor according to an embodiment of the present invention.

FIG. 11 is a view showing a state in which the porous member in FIG. 10 is separated.

FIG. 12 is a cross-sectional view taken along line C-C of FIG. 10.

FIG. 13 is a diagram showing another type of gas supply part that can be applied to a bioreactor according to an embodiment of the present invention.

FIG. 14 is a diagram schematically showing how the height of the gas supply part is changed in a bioreactor according to an embodiment of the present invention.

FIG. 15 is a diagram showing a state in which the gas supply part is connected to a sterilization filter in a bioreactor according to an embodiment of the present invention.

### DETAILED DESCRIPTION OF THE EMBODIMENTS

Hereinafter, with reference to the attached drawings, embodiments of the present invention will be described in detail so that those skilled in the art can easily practice the present invention. The present invention may be implemented in many different forms and is not limited to the embodiments described herein. In order to clearly explain the present invention in the drawings, parts that are not related to the description are omitted, and identical or similar components are assigned the same reference numerals throughout the specification.

The bioreactor 100 according to an embodiment of the present invention may culture/proliferate cells or obtain exosomes naturally secreted from cells by using a solution (r) and a support assembly 120 in a state where the support assembly 120 is disposed together with a certain amount of the solution (r) in an inner space (S).

In the present invention, the exosome (e) is a substance that exchanges signals between cells, and it may be an endoplasmic reticulum with a size of 30 nm to 200 nm or less.

In addition, the solution filled in the inner space (S) may be a medium including nutrients that are necessary for cell culture/proliferation, or it may be a buffer solution that does not react with cells or exosomes.

In this case, the buffer solution may be mixed with the cells or exosomes, and it may be a solution that protects the cells or exosomes. As an example, the buffer solution may be known phosphate-buffered saline (PBS).

In addition, the medium may further include magnetic particles that are coated with a peptide motif.

Accordingly, when the support assembly 120 is disposed in the inner space (S) to be immersed in the medium while the inner space (S) is filled with a certain amount of medium, the bioreactor according to an embodiment of the present invention 100 may culture/proliferate cells by using nutrients supplied from the medium.

In addition, when the support assembly 120 is disposed in the inner space (S) to be immersed in the buffer solution while the inner space (S) is filled with a certain amount of buffer solution including cells, the bioreactor 100 according to an embodiment of the present invention may obtain exosomes secreted from the cells.

Herein, the cells may be adherent cells attached to a support 121 constituting the support assembly 120, or they may be floating cells floating in the medium or buffer solution.

In this case, the bioreactor 100 according to an embodiment of the present invention may supply gas with a solution filled in the inner space (S) such that the cells may be smoothly cultured/proliferated or secrete exosomes in the inner space (S) even if the inner space (S) is implemented as a closed space.

In addition, the gas supplied to the solution may be supplied to the solution (r) in a state where air bubbles are removed.

For example, the gas may be carbon dioxide, but is not limited thereto, and it may be appropriately changed depending on the type of cell. The gas may be a mixed gas in which carbon dioxide, oxygen and the like are mixed.

In addition, when the gas includes oxygen, the gas may be a mixed gas with an oxygen concentration of 2 to 14%.

Through this, the gas supplied from the outside to the inner space (S) in the bioreactor 100 according to an embodiment of the present invention may be dissolved in the solution filled in the inner space (S).

As a result, the solution filled in the inner space (S) may be maintained in a state that is suitable for culturing or survival of cells through the dissolution of the gas even as time passes. For example, the solution filled in the inner space (S) may be maintained at an appropriate pH required for cell culture through the dissolution of the gas.

In addition, when the gas supplied to the solution is a mixed gas with an oxygen concentration of 2 to 14%, the bioreactor 100 according to an embodiment of the present invention may maintain the inner space (S) in a low oxygen environment similar to the human body. Therefore, cells or exosomes obtained through the bioreactor 100 according to an embodiment of the present invention may be in a state similar to human cells or exosomes.

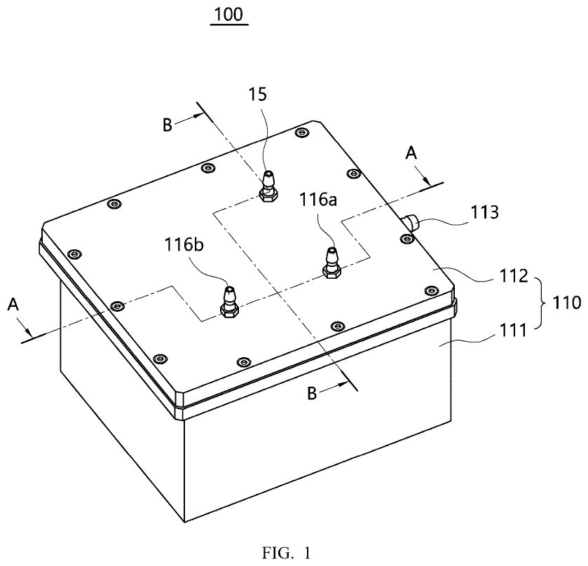

To this end, the bioreactor 100 according to an embodiment of the present invention may include a housing 110, a support assembly 120 and a gas supply part 130, as shown in FIGS. 1 to 4.

The housing 110 may be formed in the shape of a container with an inner space (S) to accommodate the support assembly 120 and the solution therein.

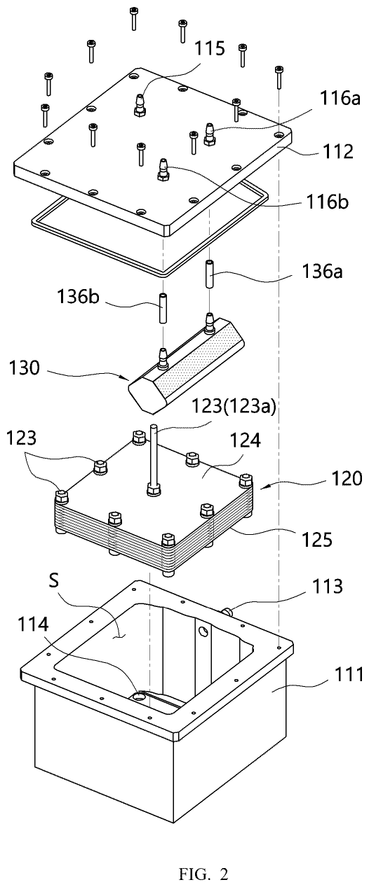

For example, as shown in FIG. 2, the housing 110 may include a housing body 111 in the shape of a container having an inner space (S) with an open upper surface, and a cover 112 configured to cover an open upper part of the housing body 111.

In this case, the housing body 111 may include a port 113 provided to communicate with the inner space (S).

Through this, the inner space (S) may be filled with a solution supplied from the outside through the port 113, and the solution filled in the inner space (S) may be discharged to the outside through the port 113.

For example, when the bioreactor 100 according to an embodiment of the present invention is used to culture/proliferate cells, the port 113 may serve as a seeding port that supplies a medium including cells to be cultured from the outside to the inner space (S) such that cells to be cultured can be seeded in the inner space (S).

In addition, when the bioreactor 100 according to an embodiment of the present invention is used for culturing cells, the port 113 may also serve as a harvesting port that discharges a medium including cells from the inner space (S) to the outside such that cells that have been cultured in the inner space (S) can be harvested.

As another example, when the bioreactor 100 according to an embodiment of the present invention is used to obtain exosomes secreted from cells, the port 113 may serve as a supply port that supplies a buffer solution to the inner space (S).

In addition, when the bioreactor 100 according to an embodiment of the present invention is used to obtain exosomes secreted from cells, the port 113 may also serve as an exhaust port that discharges a buffer solution including exosomes from the inner space (S) to the outside such that exosomes included in the buffer solution can be obtained.

In this case, the bioreactor 100 according to an embodiment of the present invention may include a vent 115 formed in the housing 110. Such a vent 115 may be formed in the housing 110 so as to communicate with the inner space (S).

Such a vent 115 may control the internal pressure of the inner space (S) by allowing air existing in the inner space (S) to be discharged to the outside during a process of supplying a solution to the inner space (S) or discharging a solution filled in the inner space (S) to the outside.

Accordingly, even if a solution is supplied from the outside to the inner space (S) through the port 113, the internal pressure of the inner space (S) may be controlled through the vent 115, thereby preventing bubbles from being generated in the inner space (S) during a process of injecting the solution into the inner space (S) through the port 113.

Meanwhile, the inner space (S) may be divided into a first space (S1) in which the support assembly 120 is disposed and a second space (S2) in communication with the port 113 via a virtual boundary line (L).

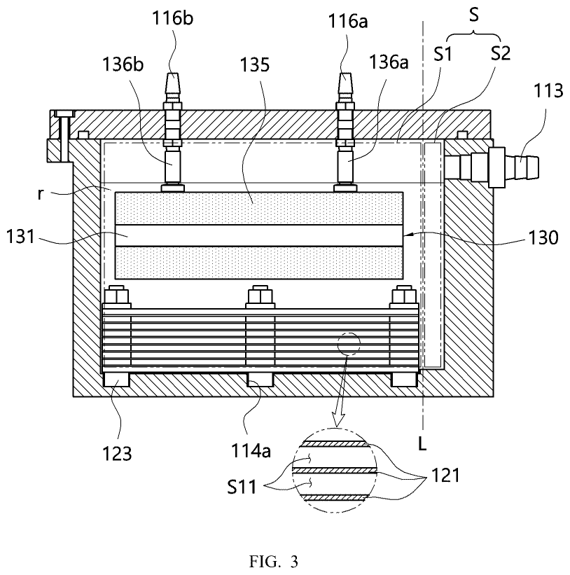

That is, as shown in FIG. 3, the inner space (S) may include a first space (S1) in which the support assembly 120 is disposed, and a second space (S2) formed between an inner surface of the housing 110, in which the port 113 is formed, and the support assembly 120.

In this case, the first space (S1) may be a space where the support assembly 120 is disposed, and the second space (S2) may be a space adjacent to the first space (S1) in which the support assembly 120 is not disposed.

In addition, the second space (S2) may be a buffer space where the solution supplied from the outside through the port 113 stays before flowing into each compartment space (S11).

Herein, the compartment space (S11) may be a space defined by two supports 121 constituting the support assembly 120 in the first space (S1).

Accordingly, after disposing the bioreactor 100 according to an embodiment of the present invention such that the port 113 is facing downward, if a solution is supplied from the outside through the port 113, the solution supplied from the outside may fill some of the first space (S1) after filling the second space (S2).

Next, if the bioreactor 100 according to an embodiment of the present invention is changed such that the port 113 faces upward, the solution filling some of the spaces among the second space (S2) and the first space (S1) may move to each compartment space (S11).

In this case, the support assembly 120 may be disposed to be immersed in a solution in the first space (S1), and the first space (S1) may be divided into a plurality of compartment spaces (S11) through a plurality of supports 121 that are disposed in multiple stages with intervals along one direction.

Accordingly, in the bioreactor 100 according to an embodiment of the present invention, since the first space (S1) filled with the solution may be divided into a plurality of compartment spaces (S11) in which cells can be cultured/proliferated, if the cells are floating cells, the frequency of contact between the floating cells floating in the solution and the support 121 may be increased.

Through this, the bioreactor 100 according to an embodiment of the present invention allows floating cells to exist in the compartment space (S11) defined through the two supports 121, and thus, the contact frequency between the floating cells and the support 121 may be increased.

In other words, compared to a conventional backpack-type bioreactor, the bioreactor 100 according to an embodiment of the present invention may allow floating cells to float within a limited volume of the compartment space (S11) through two supports 121 even when the number of floating cells included in a solution per unit area or per unit volume is the same. Accordingly, the floating cells included in the solution per unit area or per unit volume may more frequently come into contact with one surface of each of the two supports 121 that are disposed at the upper and lower parts within a limited volume of the compartment space (S11) through the two supports 121.

As a result, the bioreactor 100 according to an embodiment of the present invention may increase the frequency of contact between floating cells included in the solution and the support 121 such that the floating cells can grow more smoothly through frequent cell-to-cell interaction and secrete a larger amount of exosomes.

The support assembly 120 may be disposed in the first space (S1) as described above.

Such a support assembly 120 may be configured to increase the degree of integration of a plurality of supports 121, and when the cells are floating cells, it may be configured to increase the frequency of contact with the floating cells as described above.

Through this, the bioreactor 100 according to an embodiment of the present invention may smoothly culture/proliferate a large amount of cells and obtain a large amount of exosomes secreted from the cells.

For example, the support assembly 120 may be configured as a stacked structure in which a plurality of supports 121 are disposed to be in parallel and spaced apart along the height direction of the housing 110.

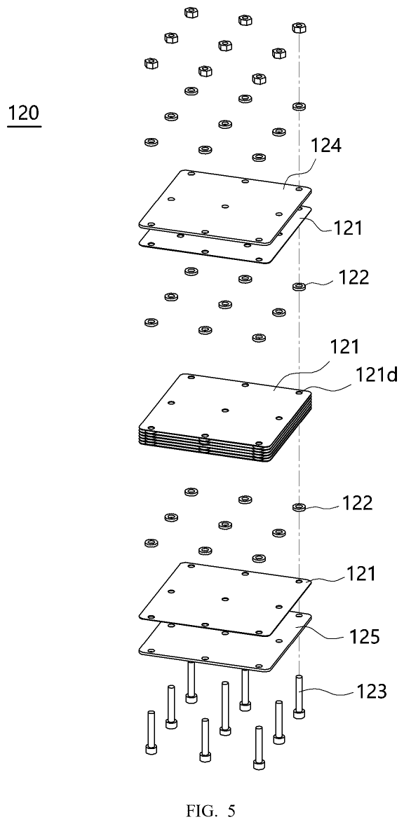

As a specific example, as shown in FIGS. 2 and 5, the support assembly 120 may include a plurality of fastening bars 123 having a predetermined length and a plurality of spacers 122 fastened to the fastening bars 123, and the plurality of support members 121 may be fitted into each of the fastening bars 123.

Herein, as shown in FIG. 5, the spacer 122 may be a ring-shaped member such that it can be fastened one to one with one fastening bar 123.

In this case, the plurality of fastening bars 123 may be spaced apart from each other at a predetermined distance, and the plurality of fastening bars 123 may be formed into a plate-shaped upper plate 124 and lower plate 125, respectively.

Accordingly, a plurality of fastening bars 123, each of which is fixed at both ends to the upper plate 124 and the lower plate 125, may be maintained while being spaced apart from each other, and the plurality of supports 121 may be arranged in multiple stages such that one side faces each other between the upper plate 124 and the lower plate 125.

In addition, the plurality of supports 121 may be respectively fastened to the fastening bars 123 through a plurality of fastening holes 121d formed through positions corresponding to the plurality of fastening bars 123.

Moreover, the plurality of spacers 122 may be respectively inserted into the plurality of fastening bars 123 like the supports 121, and the plurality of spacers 122 and the plurality of supports 121 may be alternately fastened to each fastening bar 123.

Accordingly, the spacers 122 may be respectively disposed between two supports 121 arranged along the height direction of the housing 110, and two adjacent supports 121 may maintain a spaced distance from each other through the spacer 122, and the upper plate 124, the lower plate 125, the plurality of supports 121 and the spacer 122 may be integrated through a plurality of fastening bars 123.

Through this, between the plurality of supports 121, a plurality of compartment spaces (S11) may be formed, each of which is separated by two supports 121 arranged along the height direction of the housing 110, while having the same height as the height of the spacer 122.

However, the support assembly 120 is not limited thereto, and various known methods may be applied as long as the plurality of supports 121 are arranged to be parallel to each other in one direction and may be maintained at a certain distance from each other.

In this case, the lower end of at least one fastening bar 123a among the plurality of fastening bars 123 may be in contact with a bottom surface of the inner space (S), and the upper end may be disposed to be in in contact with an inner surface of the cover 112.

For example, among the plurality of fastening bars 123, both ends of the fastening bar 123a fastened to the central part of the support assembly 120 may be disposed in the inner space (S) such that the bottom surface of the inner space (S) is in contact with the inner surface of the cover 112.

In this case, the housing body 111 and the cover 112 may include fixing grooves 114a, 114b formed inward on one surface facing each other such that both ends of the fastening bar 123a can be inserted and fixed to a certain depth.

Through this, when the support assembly 120 is disposed to be immersed in a solution filled in the inner space (S), even if buoyancy occurs in the support assembly 120, the support assembly 120 may be prevented from floating due to buoyancy through the fastening bar 123.

Herein, the fixing grooves 114a formed on the inner surface of the housing body 111 may be provided so as to correspond one-to-one with the plurality of fastening bars 123 at positions corresponding to the plurality of fastening bars 123.

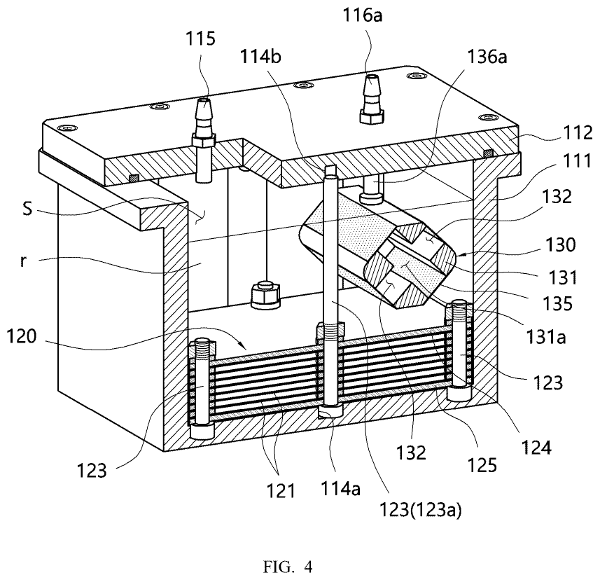

In this case, as shown in FIG. 4, the gas supply part 130 may be disposed in the inner space (S) so as to be located at a position that is not interfered with by the fastening bar 123a.

Meanwhile, the surface of the support 121 may have a surface coated with a peptide having cell proliferation properties.

That is, the surface of the support 121 may be coated with a peptide motif that has cell proliferation properties.

In addition, the support 121 may be provided in a plate shape with a predetermined area to expand the area where cells can contact or be attached.

As a result, the bioreactor 100 according to an embodiment of the present invention may increase the degree of integration of the support 121 for cell culture or proliferation and exosome secretion.

Moreover, in the bioreactor 100 according to an embodiment of the present invention, when floating cells float in the solution, a plate-shaped support 121 is disposed on the upper and lower sides of each of the plurality of compartment spaces (S11), and thus, floating cells floating in each compartment space (S11) may contact the surface of the support 121 more frequently.

Through this, the bioreactor 100 according to an embodiment of the present invention may culture/proliferate a larger amount of cells through one culture, and the amount of exosomes secreted from the cells may increase.

Additionally, in the bioreactor 100 according to an embodiment of the present invention, floating cells floating within the compartment space (S11) may more smoothly contact the surface of the support 121 such that the floating cells can grow more smoothly in each separate compartment space (S11).

Moreover, in the bioreactor 100 according to an embodiment of the present invention, the floating cells may promote growth and secrete a larger amount of exosomes by allowing cell-to-cell interactions and peptide signals to occur more frequently in each separate compartment (S11).

For this purpose, the support 121 may include a nanofiber membrane 121a in which nanofibers are formed into a three-dimensional network structure through electrospinning, and the nanofiber membrane 121a may be provided in a pair to form both surfaces of the support 121.

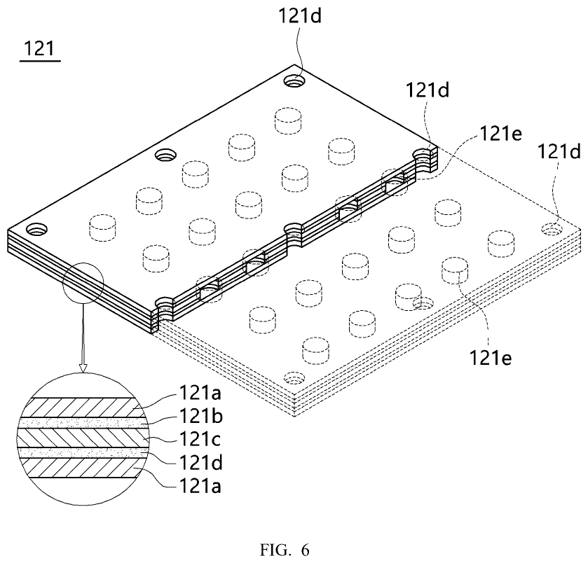

As an example, as shown in FIG. 6, the support 121 may have a five-layer structure including a pair of nanofiber membranes 121a attached to both surfaces of the support member 121c through an adhesive layer 121b.

Herein, the support member 121c may be a plate-shaped film member and may support one surface of the nanofiber membrane 121a. Through this, even if the nanofiber membrane 121a is flexible and is formed in a plate shape with a predetermined area, the nanofiber membrane 121a may be supported through the support member 121c, thereby preventing bending or sagging.

Accordingly, each support 121 constituting the support assembly 120 may maintain an unfolded state, thereby enabling cells to be cultured smoothly.

Moreover, as described above, the nanofiber membrane 121a forming a surface of the support 121 may be a plate-shaped nanofiber membrane coated with a peptide motif having cell proliferation properties such that the surface of the support 121 can have cell proliferation properties.

In this case, in the bioreactor 100 according to an embodiment of the present invention, even if the total number of the plurality of supports 121 arranged in multiple stages to define a plurality of compartment spaces (S11) in the first space (S1) increases, the gas supplied from the gas supply part 130 may be smoothly supplied to a solution filled in each compartment space (S11) defined through the two supports 121.

To this end, as shown in FIG. 6, the support 121 may include a plurality of passage holes 121e formed through a predetermined area.

That is, the plurality of passage holes 121e serve as a passage through which the gas supplied to the solution through the gas supply part 130 passes through the support 121 and moves downward, thereby improving gas flow.

To this end, the plurality of passage holes 121e may be formed in a portion of the support 121 that does not allow gas to pass through. For example, the plurality of passage holes 121e may be formed in the support 121 to penetrate the adhesive layer 121b and the support member 121c.

In this case, the upper plate 124 may include a plurality of moving holes (not shown) formed through positions corresponding to the plurality of passage holes 121e.

Accordingly, the gas flowing into the solution filled in the inner space (S) through the gas supply part 130 may smoothly move to the lower side through the plurality of passage holes 121e formed in the support 121.

As a result, the solution filled in each compartment space (S11) may smoothly receive gas from the gas supply part 130 through the plurality of passage holes 121e regardless of location.

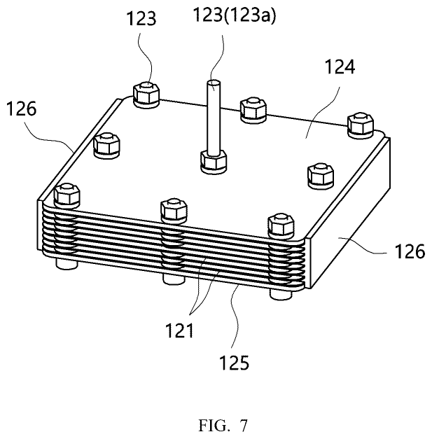

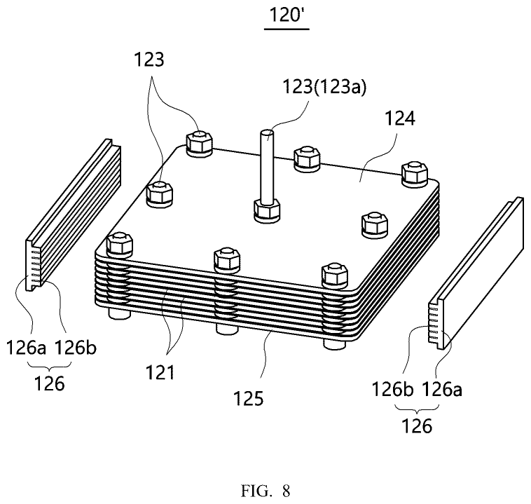

Meanwhile, in the present invention, as shown in FIGS. 7 to 9, the support assembly 120′ may further include a blocking member 126 fastened to a side surface so as to cover an open side surface of each compartment space (S11) that is separated by two supports 121.

For example, the blocking member 126 may be provided to cover both left and right-side surfaces of the support assembly 120′ with reference to FIG. 7.

Herein, the blocking member 126 may include a plate-shaped blocking body 126a having a predetermined area and a plurality of fastening grooves 126b formed inwardly at intervals along the height direction on one surface of the blocking body 126a, and an end of the support 121 may be inserted into the fastening grooves 126b.

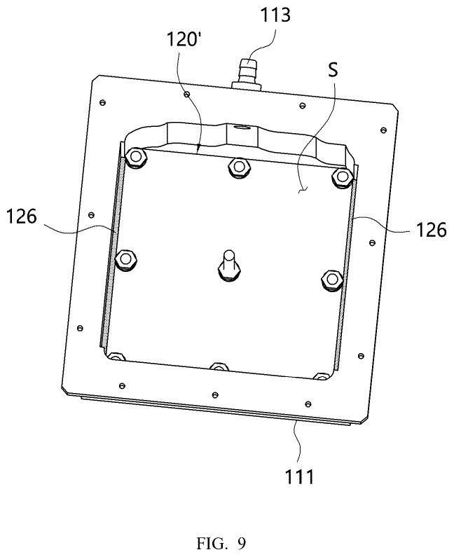

In this case, as shown in FIG. 9, the support assembly 120′ may be inserted into the housing body 111 such that the blocking member 126 does not face an inner surface of the housing body 111 where the port 113 is formed.

However, the shape of the blocking member 126 is not limited thereto, and it may be changed into various shapes depending on design conditions as long as it can cover the open side surface of the compartment space (S11) by covering a side surface of the support assembly 120′.

Accordingly, in the bioreactor 100 according to an embodiment of the present invention, when a certain amount of solution is filled in the inner space (S) and the support assembly 120′ is arranged to be immersed in the solution, each of the compartment spaces (S11) separated by two supports 121 in the first space (S1) may be sealed on both side surfaces by the blocking member 126.

As a result, each compartment space (S11) may be divided more clearly by being defined through the blocking member 126 as well as the two supports 121.

The gas supply part 130 may supply gas for cell culture/proliferation or exosome secretion from the outside to a solution filled in the inner space (S).

Through this, in the bioreactor 100 according to an embodiment of the present invention, the solution filled in the inner space (S) may maintain a suitable state for cell culture or survival through the dissolution of gas supplied from the gas supply part 130 even over time.

In this case, the gas supplied to the solution through the gas supply part 130 may have air bubbles removed, and even if the gas is supplied to the solution through the gas supply part 130, the generation of air bubbles during the gas supply process may be prevented.

Additionally, in the bioreactor 100 according to an embodiment of the present invention, the gas supply part 130 may be disposed in the inner space (S) such that the gas can be more smoothly dissolved in the solution.

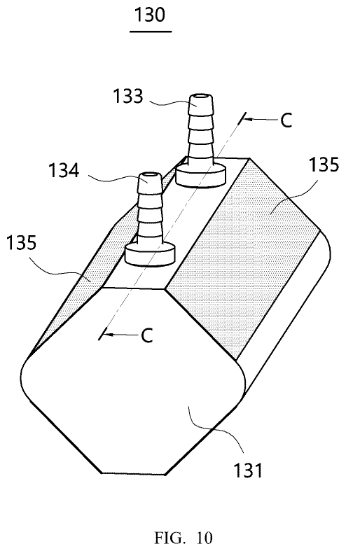

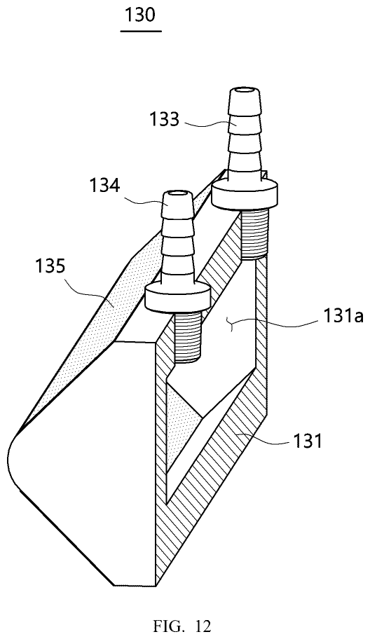

For this purpose, as shown in FIGS. 10 to 12, the gas supply part 130 may include a main body 131 having a gas storage space 131a filled with gas supplied from the outside, an opening 132 formed in the main body 131 so as to communicate with the gas storage space 131a, and a plate-shaped porous member 135 configured to surround the main body 131 to cover the opening 132.

In addition, the gas supply part 130 may further include a gas inlet 133 provided in the main body 131 such that gas can be introduced toward the gas storage space 131a, and a gas outlet 134 provided in the main body 131 such that gas in the gas storage space 131a can be discharged to the outside.

In this case, the gas inlet 133 in the gas supply part 130 may be connected to an external gas supply source (not shown), and as described above, the gas supply source may supply a type of that is gas suitable for cell culture or survival to the gas supply part 130.

Herein, the gas supplied from the gas supply source may be carbon dioxide, but the gas is not limited thereto, and it may be appropriately changed depending on the type of cell. The gas supplied from the gas supply source may be a mixed gas in which carbon dioxide, oxygen and the like are mixed. Moreover, when the gas from the gas supply source includes oxygen, the gas may be a mixed gas with an oxygen concentration of 2 to 14%.

Accordingly, the gas supplied from the gas supply source may flow into the gas storage space 131a of the main body 131, and the gas introduced into the gas storage space 131a may pass through the porous member 135 surrounding the main body 131 through the opening 132 and then move toward the solution filled in the inner space (S).

Through this, the bioreactor 100 according to an embodiment of the present invention may easily control the cell culture environment or survival environment by easily changing the type and concentration of gas supplied from the gas supply source to the gas supply part 130 even if the inner space (S) is implemented as a sealed space.

In this case, the opening 132 formed in the main body 131 may be provided to have a predetermined area, and the porous member 135 may be attached to the main body 131 so as to completely cover the opening 132.

In addition, the main body 131, like the support assembly 120, may be arranged to be immersed in the solution filled in the inner space (S).

For example, the main body 131 may be positioned on an upper side of the support assembly 120 in the inner space (S), and be disposed to be immersed in the solution.

Accordingly, the gas stored in the gas storage space 131a may have air bubbles removed while passing through the porous member 135 covering the opening 132, and be dispersed while passing through the porous member 135 having a predetermined area such that it can move toward the solution over a large area.

That is, the gas stored in the above gas storage space 131a may move toward the solution through a large area corresponding to the surface area of the porous member 135 that directly contacts the solution and corresponds to the area of the opening 132 in the inner space (S).

Through this, the bioreactor 100 according to an embodiment of the present invention may more smoothly dissolve gas supplied from the gas supply part 130 into a solution filled in the inner space (S), and even if gas is supplied from the gas supply part 130 toward the solution, the generation of bubbles may be prevented during the gas supply process.

In this case, the gas inlet 133 in the gas supply part 130 may serve as an inlet for introducing gas supplied from the gas supply source into the gas storage space 131a, and the gas outlet 134 may control the internal pressure of the gas storage space 131a by allowing gas existing in the gas storage space 131a to be discharged to the outside during the process of supplying gas to the gas storage space 131a.

Accordingly, even if gas is supplied from a gas supply source to the gas storage space 131a through the gas inlet 133, the internal pressure of the gas storage space 131a may be controlled through the gas outlet (134), thereby preventing bubbles from being generated due to excessive pressure during the process in which the gas stored in the gas storage space 131a passes through the porous member 135.

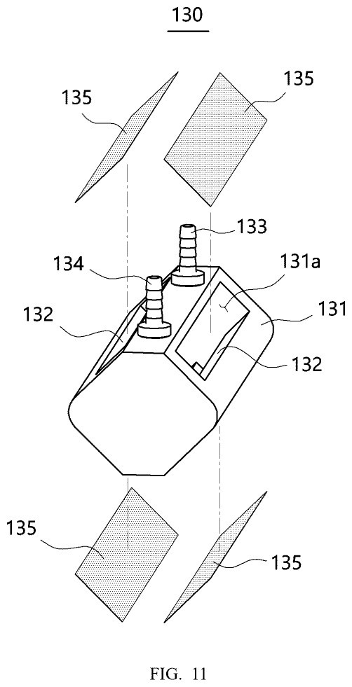

As a specific example, as shown in FIGS. 10 and 11, the main body 131 may be formed as an octahedral hollow shape with an approximately hexagonal cross-section, and the gas storage space 131a may be an inner space of the main body 131.

In addition, the opening 132 may be formed to penetrate so as to have a predetermined area in each of four surfaces forming the side surfaces among 8 surfaces of the main body 131, and the gas inlet 133 and gas outlet 134 may be provided so as to communicate with the gas storage space 131a on one surface of the main body 131 forming an upper surface among the 8 surfaces of the main body 131.

In this case, the gas inlet 133 and gas outlet 134 may be directly fastened to the housing 110 such that a portion of the length protrudes outside the housing 110, but the gas inlet 133 and gas outlet 134 may be fastened to the housing 110 through separate connecting pipes 136a, 136b.

As a non-limiting example, as shown in FIGS. 3 and 4, the housing 110 may include two fittings 116a, 116b provided on a cover 112, and the gas inlet 133 and the gas outlet 134 may be respectively fastened to the two fittings 116a, 116b through separate connecting pipes 136a, 136b.

Through this, the gas supply part 130 may be disposed in the inner space (S) in a state of being spaced apart by a certain distance from the cover 112 through the connecting pipe 136a, 136b, and the main body 131 may be disposed so as to be immersed in the solution filled in the inner space (S).

In the drawings, it is shown that there are 4 openings 132 and 4 porous members 135, but the present invention is not limited thereto, and the total numbers of openings 132 and porous members 135 may be appropriately changed depending on design conditions.

Moreover, the main body 131 may also be changed into various known shapes as long as it has a shape that includes a gas storage space 131a inside and an opening 132 formed with a predetermined area so as to communicate with the gas storage space 131a formed inside.

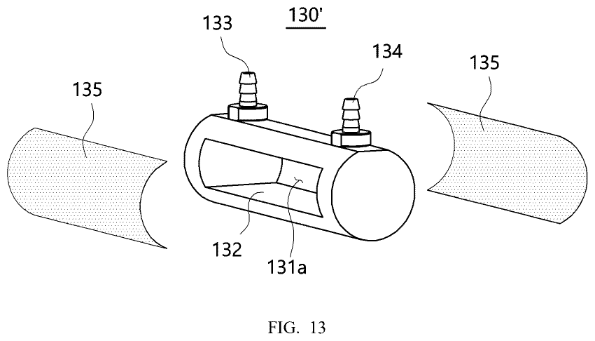

For example, as shown in FIG. 13, the main body 131 of the gas supply part 130′ may be formed in an approximately hollow cylindrical shape, and the gas storage space 131a may be an inner space of the main body 131. In addition, the main body 131 may include two openings 132 formed to penetrate a portion of the entire periphery, and two porous members 135 may be attached to the main body 131 to cover the two openings 132, respectively.

In addition, although the drawings show that the openings 132 and the porous members 135 correspond one-to-one, the present invention is not limited thereto. When a plurality of openings 132 are provided, the plurality of openings may all be covered by a single porous member, and the total number of the openings 132 and the total number of the porous members 135 may be appropriately changed.

In this case, the porous member 135 covering the opening 132 in the gas supply parts 130, 130′ may be provided to allow the gas to move from the gas storage space 131a to the solution through the opening 132 while blocking the solution in the inner space (S) from moving to the gas storage space 131a.

That is, the porous member 135 may block foreign substances and liquids from passing through, while allowing gases such as carbon dioxide to pass through.

For example, the porous member 135 may be a water-repellent nanofiber membrane. However, the material of the porous member 135 is not limited thereto, and any material that blocks solid and liquid fluids from passing through but allows gaseous fluids to pass through may be used without limitation.

Accordingly, even if gas is supplied to the solution filled in the inner space (S) from the outside through the gas supply parts 130, 130′, the solution filled in the inner space (S) may not be contaminated by other foreign substances.

In addition, since the solution filled in the inner space (S) may be blocked from moving toward the gas storage space 131a of the gas supply parts 130, 130′ through the porous member 135, the direction of movement of the gas supplied from the gas supply source to the gas storage space 131a may always be limited to the solution side surrounding the exterior of the main body 131 in the gas storage space 131a.

Accordingly, in the bioreactor 100 according to an embodiment of the present invention, the flow direction of gas may be maintained constant between the gas storage space 131a and the solution, and thus, even if gas passes through the porous member 135 from the above gas storage space 131a, the possibility of bubbles being generated during the process of passing through the porous member 135 may be fundamentally prevented.

Meanwhile, the gas supply parts 130, 130′ may be coupled to the housing 110 such that the height of the main body in the inner space (S) can be adjusted.

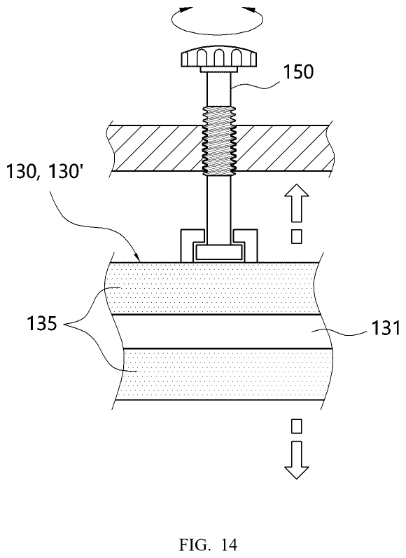

For example, as shown in FIG. 14, the housing 110 may include an adjustment bar 150 having a predetermined length so as to pass through the cover 112, and the adjustment bar 150 may be rotatably screw-coupled to the cover 112.

In this case, one end of the adjustment bar 150 may be connected to one side of the main body 131.

Through this, when the adjustment bar 150 is rotated, the height of the main body 131 may be adjusted in the inner space (S).

Accordingly, by changing the height of the gas supply part 130 disposed in the inner space (S) depending on the amount of solution filled in the inner space (S) or the height of the housing 110, the main body 131 may always be maintained in a state of being immersed in the solution regardless of the amount of solution filled in the inner space (S).

Meanwhile, the bioreactor 100 according to an embodiment of the present invention may further include a sterilization filter 140 connected to the gas inlet 133 of the gas supply parts 130, 130′.

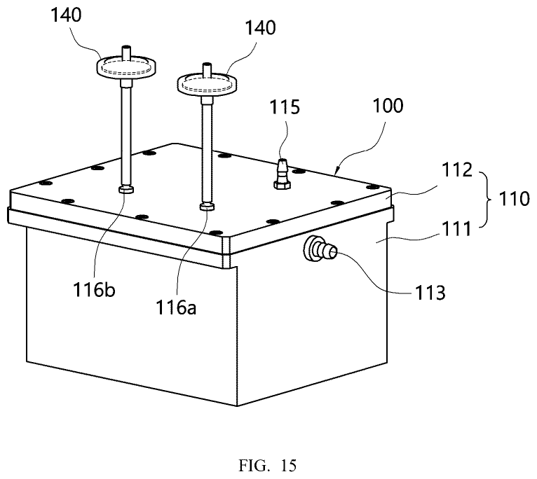

For example, as shown in FIG. 15, the sterilization filter 140 may be provided to be connected to fittings 116a, 116b provided in the housing 110.

Accordingly, the gas supplied from the gas supply source may be supplied to the gas supply parts 130, 130′ after passing through the sterilization filter 140.

As a result, since sterile gas may always be introduced into the gas storage space 131a, contamination of the solution filled in the inner space (S) by bacteria or microorganisms may be prevented in advance.

For example, the sterilization filter 140 may be a known sterilizing syringe filter, but is not limited thereto. If the gas supplied from the gas supply source may be supplied to the gas supply part 130 in a sterilized state, various known sterilization filters may be employed.

Although one embodiment of the present invention has been described above, the spirit of the present invention is not limited to the embodiment presented in the present specification, and those skilled in the art who understand the spirit of the present invention will be able to easily suggest other embodiments by modifying, changing, deleting or adding components within the scope of the same spirit, but this will also be considered to fall within the spirit of the present invention.

What is claimed is:

1. A bioreactor for culturing/proliferating cells or acquiring exosomes secreted from the cells, comprising:
a housing in a shape of a container having an inner space that is filled with a predetermined amount of a solution;
a support assembly that comprises a plurality of supports formed of a plate-shaped member having a predetermined area and spaced apart from each other in one direction, and is disposed in the inner space to be immersed in the solution; and
a gas supply part that comprises a main body having a gas storage space filled with gas supplied from an outside, and is disposed in the inner space to supply gas from the gas storage space to the solution;
wherein the main body is disposed to be immersed in the solution filled in the inner space such that the gas stored in the gas storage space moves to the solution and is dissolved.

2. The bioreactor of claim 1, wherein the gas supply part comprises a main body having a gas storage space filled with gas supplied from the outside, an opening formed in the main body so as to communicate with the gas storage space, a plate-shaped porous member surrounding the main body to cover the opening, a gas inlet provided in the main body so as to allow gas to flow into the gas storage space, and a gas outlet provided in the main body so as to allow gas in the gas storage space to be discharged to the outside.

3. The bioreactor of claim 2, wherein the plate-shaped porous member is provided with a hydrophobic membrane so as to allow the gas to move from the gas storage space to the solution through the opening while blocking a solution in the inner space from moving to the gas storage space.

4. The bioreactor of claim 2, wherein the bioreactor further comprises a sterilization filter that is connected to the gas inlet.

5. The bioreactor of claim 2, wherein the gas supply part comprises a plurality of openings that are spaced apart along a peripheral direction of the main body, and the openings are formed in the main body to have a predetermined area.

6. The bioreactor of claim 1, wherein the gas supply part is disposed in the inner space such that the main body is positioned at an upper part of the support assembly.

7. The bioreactor of claim 1, wherein the gas supply part is coupled to the housing such that a height of the main body is adjusted in the inner space.

8. The bioreactor of claim 1, wherein the support comprises a plate-shaped support member having a predetermined area, and a pair of nanofiber membranes that are respectively attached to both surfaces of the support member via an adhesive layer, and
wherein the nanofiber membrane is a motif-coated plate-shaped nanofiber membrane.

9. The bioreactor of claim 8, wherein the support comprises a plurality of penetration holes formed through the support member such that gas introduced into the inner space from the gas supply part passes smoothly.

10. The bioreactor of claim 1, wherein the support assembly comprises:
an upper plate and a lower plate having a predetermined area;
a plurality of supports disposed such that one surface faces each other between the upper plate and the lower plate;
a spacer disposed between two supports facing each other so as to space the two supports apart; and
a plurality of fastening bars that fasten and integrate the upper plate, the lower plate, the plurality of supports and the spacer.

11. The bioreactor of claim 10, wherein the housing comprises a housing body in the shape of the container having the inner space with an open upper part, and a cover configured to cover the open upper part of the housing body, and
wherein at least any one of the plurality of fastening bars is positioned in the inner space such that a lower end contacts a bottom surface of the inner space and an upper end contacts an inner surface of the cover, thereby preventing the support assembly from floating in the inner space.

12. The bioreactor of claim 1, wherein the bioreactor further comprises a port formed in the housing so as to supply a solution to the inner space or discharge a solution in the inner space to the outside, and a vent formed in the housing so as to communicate with the inner space.

13. The bioreactor of claim 1, wherein the gas is a mixed gas with an oxygen concentration of 2 to 14%.

14. The bioreactor of claim 1, wherein the solution is a medium or buffer solution.
